(19) United States
(12) Patent Application Publication
(10) Pub. No.: US 2021/0391063 A1
(43) Pub. Date: Dec. 16, 2021

(54) SYSTEM AND METHOD FOR DYNAMIC WORKLOAD BALANCING BASED ON PREDICTIVE ANALYTICS

(71) Applicant: KONINKLIJKE PHILIPS N.V., EINDHOVEN (NL)

(72) Inventors: Lasith Adhikari, Cambridge, MA (US); David Paul Noren, Cambridge, MA (US); Gregory Boverman, Cambridge, MA (US); Qianxi Li, Cambridge, MA (US)

(21) Appl. No.: 17/169,710

(22) Filed: Feb. 8, 2021

Related U.S. Application Data
(60) Provisional application No. 63/039,053, filed on Jun. 15, 2020.

(51) Int. Cl. G16H 40/20; G06N 20/00; G16H 50/20; G16H 10/60; G16H 50/70; G16H 40/67; A61B 5/00

(52) U.S. Cl. CPC G16H 40/20; G06N 20/00; G16H 50/20; A61B 5/746; G16H 50/70; G16H 40/67; A61B 5/7267; G16H 10/60

(57) ABSTRACT

A method for allocating resources comprising: (i) receiving information about a plurality of patients being monitored by a plurality of healthcare professionals; (ii) receiving information about a monitoring load for each of the plurality of healthcare professionals; (iii) classifying, by a trained monitoring liability classifier, each of the plurality of patients into one of a plurality of monitoring liability classes; (iv) determining a distribution of the plurality of patients for monitoring among the plurality of healthcare professionals based on both the received monitoring load for each of the plurality of healthcare professionals and the monitoring liability class for each of the plurality of patients, wherein the distribution optimizes the monitoring load for each of the plurality of healthcare professionals; and (v) redistributing the plurality of patients for monitoring among the plurality of healthcare professionals according to the determined distribution.

### FIELD OF THE DISCLOSURE

The present disclosure is directed generally to methods and systems for dynamically allocating a plurality of remotely monitored patients among a plurality of healthcare professionals.

### BACKGROUND

Centralized monitoring of non-critically ill cardiac patients from a remote facility—Central Monitoring Unit (CMU)—is no longer a new concept in healthcare. More than 60% of U.S. hospitals have been using CMUs for at least a decade. Specially trained telemetry monitoring technicians in CMUs are responsible for both interpreting cardiac rhythms and responding to alarms with minimal delay. Monitoring 24 to 60 patients at a time with 350 to 700 alarms per patient per day for 8 to 12 hours continuously is a challenging and stressful job, and life-threatening events might be missed due to fatigue or stress. Timeliness of response to life-threatening telemetry alarms is critical to improving the quality of centralized monitoring. An audit conducted by Philips Healthcare found out that on average four alarms are received by a telemetry charge nurse every minute. Also, during interviews telemetry staff often report that some telemetry technicians are responding to more alarms than others. Moreover, false or nonactionable alarms may limit a technician's ability to identify and prioritize alarm signals in a timely manner, as the signal of interest may be buried in a sea of noise. This alarm burden directly impacts technicians, leading to annoyance, anxiety, low job satisfaction, and burn-out. Currently, there is no standard patient-to-technician ratio for CMUs and the number of patients monitored by a technician is heavily dependent on his or her level of skill and experience.

Eventually, alarm fatigue can lead technicians to ignore alarms, disable alarms inappropriately, or delay a response. All of these actions could pose a significant threat to patient safety, including prolonging hospital stays and healthcare costs, and in the most serious cases leading to adverse clinical outcomes or even patient death.

The literature reports that the increase in alarm-related workload for technicians is one of the major contributors to the aforementioned unwanted outcomes, and thus for example the Alarm Management Compendium by the National Coalition for Alarm Management Safety has recommended developing profiles for frequently seen populations to reduce alarm burden. Many technology-based solutions including logic algorithms, filtering methods, or customization of alarm limits have been introduced to reduce alarm fatigue, while some studies have found that changing default alarm settings and education about cardiac monitor use are insufficient to reduce alarm fatigue. Unfortunately, these approaches have largely been unable to reduce the unwanted outcomes of alarm fatigue.

### SUMMARY OF THE DISCLOSURE

There is a continued need for methods and systems that more evenly distribute patients and their respective alarm load among monitoring healthcare professionals in order to reduce or prevent alarm fatigue and lead to improved patient outcomes.

The present disclosure is directed at inventive methods and systems for resource allocation using a resource allocation system. Various embodiments and implementations herein are directed to a system or method that receives information about patients and about the monitoring load of each of the healthcare professionals monitoring the patients. The system uses the information about the patients to classify an expected or predicted monitoring load for each patient, such as the number and/or severity of expected or predicted alarms. Using the classifications, the system distributes the patients for monitoring among the healthcare professionals that optimizes the professionals' monitoring load. The system then redistributes the patients for monitoring among the healthcare professionals according to the determined distribution.

Generally, in one aspect, a method for allocating resources using a resource allocation system is provided. The method includes: (i) receiving, by the resource allocation system, information about a plurality of patients being monitored by a plurality of healthcare professionals; (ii) receiving, by the resource allocation system, information about a monitoring load for each of the plurality of healthcare professionals; (iii) classifying, by a trained monitoring liability classifier of the resource allocation system, each of the plurality of patients into one of a plurality of monitoring liability classes; (iv) determining, by an assignment module of the resource allocation system, a distribution of the plurality of patients for monitoring among the plurality of healthcare professionals based on both the received monitoring load for each of the plurality of healthcare professionals and the monitoring liability class for each of the plurality of patients, wherein the distribution optimizes the monitoring load for each of the plurality of healthcare professionals; and (v) redistributing, by the assignment module of the resource allocation system, the plurality of patients for monitoring among the plurality of healthcare professionals according to the determined distribution.

According to an embodiment, the method further includes: receiving new information about the plurality of patients and/or the monitoring load for each of the plurality of healthcare professionals; determining an updated distribution of the plurality of patients for monitoring among the plurality of healthcare professionals based on the received new information; and redistributing the plurality of patients based on the determined updated distribution.

According to an embodiment, the method further includes: receiving, by the resource allocation system, input data comprising medical information about a plurality of monitored patients, the information comprising a monitoring liability for each of the plurality of monitored patients; generating, by a feature processing module of the resource allocation system, a plurality of features related to monitoring liability for each of the plurality of monitored patients; and training, using the plurality of features, a computer learning classifier to classify a patient in one of a plurality of monitoring liability classes, based at least in part on a predicted monitoring liability for the patient.

According to an embodiment, the information about a plurality of patients being monitored by a plurality of healthcare professionals comprises one or more of demographic information, health history, treatment data, telemetry data, and/or diagnosis data for one or more of the plurality of patients.

According to an embodiment, the information about a monitoring load for each of the plurality of healthcare professionals comprises one or more of a number of patients being monitored by the healthcare professional, and/or an experience level of the healthcare professional for one or more of the healthcare professionals.

According to an embodiment, monitoring liability comprises an expected or predicted number of alarms for a patient during a time period. According to an embodiment, monitoring liability comprises a severity of one or more of an expected or predicted number of alarms for a patient during a time period.

According to an embodiment, classifying comprises a prediction of a monitoring liability for at least one of the plurality of patients.

According to an embodiment, the plurality of monitoring liability classes comprises at least a low monitoring liability class and a high monitoring liability class.

According to another aspect is a resource allocation system. The system comprises: data comprising information about a plurality of patients being monitored by a plurality of healthcare professionals; data comprising information about a monitoring load for each of the plurality of healthcare professionals; a monitoring liability classifier trained to classify each of the plurality of patients into one of a plurality of monitoring liability classes; and a processor configured to: (i) direct the classifier to classify each of the plurality of patients into one of the plurality of monitoring liability classes; (ii) determine a distribution of the plurality of patients for monitoring among the plurality of healthcare professionals based on both the received monitoring load for each of the plurality of healthcare professionals and the monitoring liability class for each of the plurality of patients, wherein the distribution optimizes the monitoring load for each of the plurality of healthcare professionals; and (iii) redistribute the plurality of patients for monitoring among the plurality of healthcare professionals according to the determined distribution.

According to an embodiment, the processor is further configured to: receive new information about the plurality of patients and/or the monitoring load for each of the plurality of healthcare professionals; determine an updated distribution of the plurality of patients for monitoring among the plurality of healthcare professionals based on the received new information; and redistribute the plurality of patients based on the determined updated distribution.

According to an embodiment, the processor is further configured to: receive input data comprising medical information about a plurality of monitored patients, the information comprising a monitoring liability for each of the plurality of monitored patients; generate a plurality of features related to monitoring liability for each of the plurality of monitored patients; and train the classifier to classify a patient in one of a plurality of monitoring liability classes, based at least in part on a predicted monitoring liability for the patient.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for allocating resources using a resource allocation system, in accordance with an embodiment.

FIG. 2 is a flowchart of a method for generating a trained classifier, in accordance with an embodiment.

FIG. 3 is a flowchart of a method for allocating resources using a resource allocation system, in accordance with an embodiment.

FIG. 4 is a flowchart of a method for allocating resources using a resource allocation system, in accordance with an embodiment.

FIG. 5 is a schematic representation of a resource allocation system, in accordance with an embodiment.

### DETAILED DESCRIPTION OF EMBODIMENTS

The present disclosure describes various embodiments of a system and method for dynamically allocating a plurality of monitored patients among a plurality of healthcare professionals. Applicant has recognized and appreciated that it would be beneficial to provide a method and system that can more evenly distribute patients and their respective alarm load among monitoring healthcare professionals in order reduce or prevent alarm fatigue and lead to improved patient outcomes. The system receives information about patients and about the monitoring load of each of the healthcare professionals monitoring the patients. The system uses the information about the patients to classify an expected or predicted monitoring load for each patient, such as the number and/or severity of expected or predicted alarms. Using the classifications, the system distribution of the patients for monitoring among the healthcare professionals that optimizes the professionals' monitoring load. The system then redistributes the patients for monitoring among the healthcare professionals according to the determined distribution.

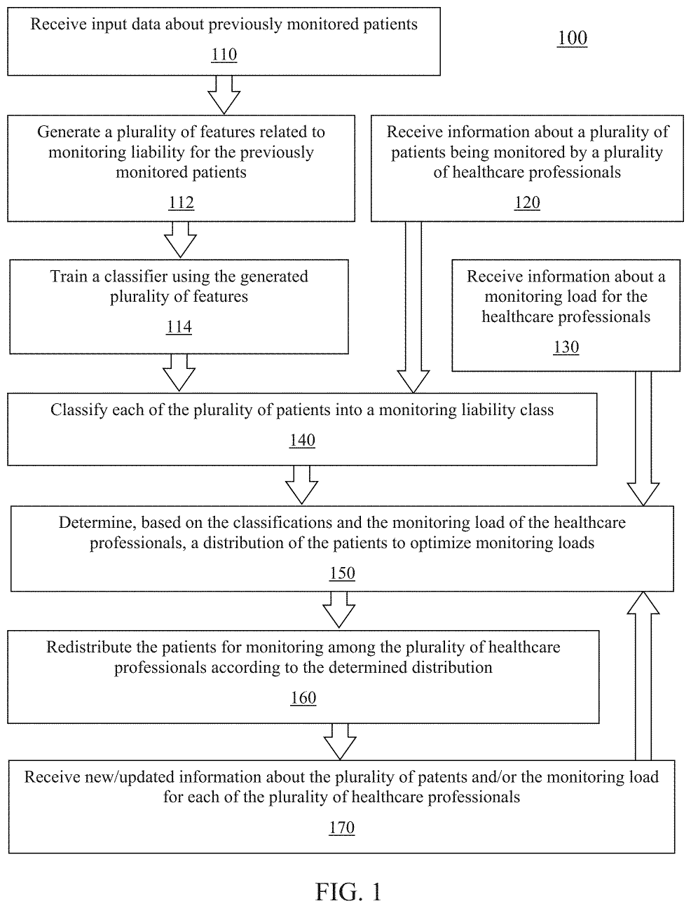

Referring to FIG. 1, in one embodiment, is a flowchart of a method 100 for allocating resources using a resource allocation system. The resource allocation system may be any resource allocation system described or otherwise envisioned herein.

As discussed in greater detail herein, the resource allocation system comprises a trained monitoring liability classifier configured to classify a patient into one of a plurality of monitoring liability classes ranging from, for example, low monitoring liability to high monitoring liability. Monitoring liability may be a prediction or expectation of certain monitoring outcomes, including but not limited to event rate or frequency, event severity, or other outcomes where an event may be an alarm or other triggering moment. ‘Low monitoring liability’ may, for example, be a prediction or expectation of monitoring outcomes below a certain threshold or specific number of outcomes, while ‘high monitoring liability’ may, for example, be a prediction or expectation of monitoring outcomes above a certain threshold or specific number of outcomes. Other classes may comprise a range of expected or predicted monitoring outcomes for a patient for a specific period of time. Depending on the needs of the resource allocation system, there can be a specific number of classifications. Alternatively, the classification may be simply a predicted or expected number and/or severity of certain monitoring outcomes.

As just a few examples of a large number of possible classifications, the classification may be one or more of: (1) fewer than X number of monitoring outcomes Y expected per time period Z; (2) more than X number of monitoring outcomes Y expected per time period Z; (3) between X₁ and X₂ number of monitoring outcomes Y expected per time period Z; (4) X number of monitoring outcomes Y expected per time period Z; and many, many more.

In some embodiments, the monitoring liability classifier is created and/or trained for the resource allocation system. Broadly, the system receives and extracts electronic medical records (e.g., demographics, diagnosis, physiologic measures, telemetry alarm data, and many other possibilities) from a hospital data repository, IT system, and/or other databases and performs variable generation and data pre-processing. For example, alarm data can be used to generate outcome variables to be used to train the classifier.

Accordingly, at step 110 of the method, the system receives input data comprising medical information about a plurality of monitored patients. The medical information includes at least the monitoring liability for each of the monitored patients. The input data may be stored in and/or received from one or more databases. The database may be a local and/or remote database. For example, the monitoring center or resource allocation system may comprise a database of input data such as a medical health record database among many other types of databases.

In order to train the classifier, the input data can comprise a wide variety of input types in addition to the monitoring liability of a patient. As an example, the input data for modelling can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or telemetry alarm related data such as arrhythmia-related alarms (ventricular tachycardia, ventricular bradycardia, atrial fibrillation, asystole, and more), physiologic alarms such as heart rate, respiratory rate, apnea, SpO₂, invasive arterial pressure, noninvasive blood pressure, and more, and/or technical alarms such as artifact, ECG leads fail, respiratory leads fail, blood pressure sensor fail, and more. Many other types of input data are possible. According to an embodiment, an initial classification made by a trained classifier can be based entirely or substantially on patient characteristics, while a subsequent classification made by the trained classifier can comprise the patient's monitoring data for a period of time since the initial classification. Many other embodiments are possible.

At step 112 of the method, a feature processing module of the resource allocation system utilizes the input data to generate a plurality of features related to monitoring liability for each of the plurality of previously monitored patients. This can be accomplished by a variety of embodiments for feature identification, extraction, and/or processing.

According to an embodiment, the resource allocation system comprises a data transformer or transformer module that reads raw input data and generates predictor and outcome variables. As an example, alarm rates at the patient level can be used as the outcome variable for model training.

According to an embodiment, the resource allocation system comprises a data pre-processor or data processing module that processes the predictor and outcome variables using a set of one or more automated rules. For example, the data pre-processor can process or clean the clinical variables using expert opinion and medical knowledge in which, for example, extreme values in physiological variables can be removed. As another example, the data pre-processor can identify and remove outliers among the variables, such as replacing the top and bottom 1% of data using random uniform values generated from the neighboring data, among other options.

According to an embodiment, the resource allocation system comprises missing data imputation or an imputation module that performs missing data imputation. For example, according to just one embodiment, the system can replace missing nominal variables with distinct ‘missing’ category and missing continuous variables will be replaced by median values. Data imputation may be in whole or in part dependent upon the classification model utilized for the resource allocation system.

The outcome of the feature processing module of the resource allocation system is a set of features related to monitoring liability for each of the plurality of previously monitored patients, which thus comprises a training data set that can be utilized to train the classifier.

At step 114 of the method, the training data set is utilized to train the classifier to classify a patient in one of a plurality of monitoring liability classes ranging from low monitoring liability to high monitoring liability, based at least in part on a predicted monitoring liability for the patient.

According to an embodiment, a machine learning classifier such as random forest, XGBoost, and/or any other classifier can be used to learn the patient clinical phenotypes and their most likely alarm rates. According to just one embodiment, hyper parameters in the machine learning classifier can be tuned using grid search technique with five-fold cross validation on 70% training data. Models can then be validated using the remaining 30% test data and the final model parameters will be selected based on performance matrices such as area under receiver operating characteristic curve and area under the precision-recall curve, among other approaches. These percentages can of course vary considerably.

Following step 114, the resource allocation system comprises a trained classifier than can be utilized to classify patients to be monitored or currently monitored rather than previously monitored patients. The trained classifier can be static such that it is trained once and is utilized for classifying. According to another embodiment, the trained classifier can be more dynamic such that it is updated or re-trained using subsequently available training data. The updating or re-training can be constant or can be periodic.

With a trained classifier, the resource allocation system is ready to classify patients and provide optimized resource allocation. Accordingly, at step 120 of the method, the resource allocation system receives information about a plurality of patients being monitored by a plurality of healthcare professionals. The information may comprise any information about the plurality of patients, including but not limited to medical information which will be informative for monitoring liability. Preferably, the received information comprises input data related to the training data for the trained classifier utilized by the system, to maximize the accuracy of the classifier. The information may be received from a local and/or remote database. The database may be a medical health record database, and/or any other database comprising the necessary information. According to an embodiment, the received information may be processed by the system such that it can be utilized by the trained classifier. For example, the system may identify, extract, and process one or more features for each patient for use by the classifier. Features may be utilized by the classifier immediately or may be stored for downstream or later use by the system.

At step 130 of the method, the resource allocation system receives information about a monitoring load for each of the plurality of healthcare professionals at the monitoring center. Notably, the monitoring healthcare professionals and the monitoring center or location may be local to and/or remote from the plurality of patients. The information may comprise any information about the monitoring healthcare professionals, including but not limited to information about the number of patients a professional is currently monitoring, the professional's current workload, the professional's current shift info, the professional's experience, the professional's scheduling preferences, the center's staffing targets or other information about the monitoring center, and much more.

The information about a monitoring load for each of the plurality of healthcare professionals at the monitoring center may be received from a local and/or remote database. The database may be a monitoring center database, and/or any other database comprising the necessary information. According to an embodiment, the received information may be processed by the system such that it can be utilized by an assignment module of the resource allocation system. For example, the system may identify, extract, and process one or more features for each monitoring professional. Features may be utilized by the assignment module immediately or may be stored for downstream or later use by the system.

At step 140 of the method, the system the trained monitoring liability classifier of the resource allocation system classifies one or more of the plurality of patients into a monitoring liability class. According to one embodiment, the classifier is trained to classify a patient into one of a plurality of different possible monitoring liability classes. For example, the plurality of different possible monitoring liability classes may comprise classes ranging from low monitoring liability to high monitoring liability, among other options. Monitoring liability may be a prediction or expectation of certain monitoring outcomes, including but not limited to event rate or frequency, event severity, or other outcomes where an event may be an alarm or other triggering moment. ‘Low monitoring liability’ may, for example, be a prediction or expectation of monitoring outcomes below a certain threshold or specific number of outcomes, while ‘high monitoring liability’ may, for example, be a prediction or expectation of monitoring outcomes above a certain threshold or specific number of outcomes. Other classes may comprise a range of expected or predicted monitoring outcomes for a patient for a specific period of time. Depending on the needs of the resource allocation system, there can be a specific number of classifications. Alternatively, the classification may be simply a predicted or expected number and/or severity of certain monitoring outcomes.

According to just one embodiment, the classifier reads pre-processed variables and predicts the alarm burden for each patient. For example, one approach may be discrete classification, which can comprise classifying patients into three classes based on their clinical and personal profiles: low, medium, and high. In particular, low class represents lower alarm burden while high represents higher alarm burden. Alternatively, the classifier may use a regression algorithm to predict the rate of alarms for a variety of classes and severities of alarms: respiration rate alarms, SpO₂ alarms, various types of heart rate and heart rhythm alarms. The burden of one type of monitoring event may be very different than another, which can be determined, for example, by interaction with skilled technicians among other approaches.

At step 150 of the method, an assignment module of the resource allocation system determines a distribution of the plurality of patients for monitoring among the plurality of healthcare professionals based on both the received monitoring load for each of the plurality of healthcare professionals and the monitoring liability class for each of the plurality of patients. The assignment module is configured to optimize the monitoring load for each of the plurality of healthcare professionals.

According to just one embodiment, the assignment module uses predicted alarm burden classes generated by the patient classification module and optimize the distribution of patients among telemetry technicians. The data about tele-ward and technicians—such as number of patients currently monitoring, current workload, current shift info, experience, and others—can also be used as inputs to optimize scheduling.

According to one embodiment, the assignment module can be configured such that all monitoring technicians have the same capability and thus can receive the same monitoring liability amount or workload. According to another embodiment, the assignment module can be configured such that one or more monitoring technicians have more or less experience or workload tolerance, and thus the monitoring liability amount or workload assigned to a monitoring technician can be in part or in whole dependent upon that additional information.

According to another embodiment, the assignment module can be static, such that assignments are made once for a monitoring technician, and/or once for a monitoring technician's shift, and/or once for a patient, among other static assignments. Alternatively, the assignment module can be dynamic such that assignments are updated or re-trained using subsequently available patient classification and/or monitoring technician data. The updating or re-training can be constant or can be periodic. For example, the assignment module can dynamically detect technician capabilities or changing workload based on monitoring their responses to monitoring events, and can thus adjust assignments dynamically.

The resource allocation system can utilize the assignment information immediately, and/or can store the assignment information for future use, including but not limited to review and validation of the assignment decisions.

At step 160 of the method, the assignment module of the resource allocation system re-distributes the plurality of patients for monitoring among the plurality of healthcare professionals according to the determined distribution. For example, the assignment module may comprise control of patient assignments, or may send a determined distribution to a control module for making changes to a current assignment list.

According to an embodiment, the system can dynamically redistribute patients according to new information. Thus, at step 170 of the method, the system receives new information about the plurality of patients and/or the monitoring load for each of the plurality of healthcare professionals. The assignment module determines an updated distribution of the plurality of patients for monitoring among the plurality of healthcare professionals based on the received new information at step 150, and at step 160 the system can redistribute the plurality of patients based on the determined updated distribution. This dynamic redistribution can be done periodically or continuously. For example, the dynamic redistribution can be performed according to a schedule, depending upon the starting or ending of a healthcare professional's shift, the introduction or removal of a patient, and many other triggering events.

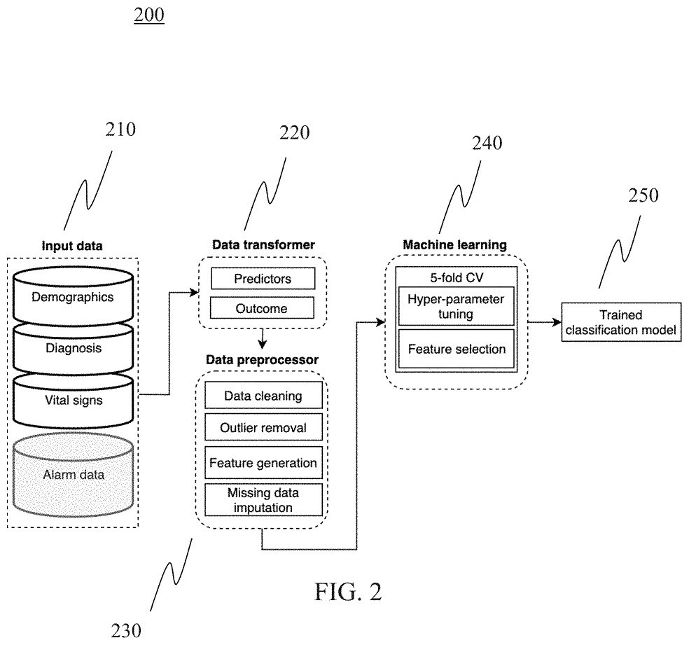

Referring to FIG. 2, in one embodiment, is a schematic representation 200 of a method for generating the trained monitoring liability classifier configured to classify a patient into one of a plurality of monitoring liability classes. At 210, the system receives input data comprising medical information about a plurality of monitored patients. The medical information includes at least the monitoring liability for each of the monitored patients. The input data may be stored in and/or received from one or more databases. The database may be a local and/or remote database. For example, the monitoring center or resource allocation system may comprise a database of input data such as a medical health record database among many other types of databases. As described above, the input data can comprise a wide variety of input types in addition to the monitoring liability of a patient.

The system utilizes the input data to generate a plurality of features related to monitoring liability for each of the plurality of previously monitored patients. This can be accomplished by a variety of embodiments for feature identification, extraction, and/or processing. For example, at 220 a data transformer or transformer module reads raw input data and generates predictor and outcome variables. As an example, alarm rates at the patient level can be used as the outcome variable for model training. At 320, a data pre-processor or data processing module processes the predictor and outcome variables using a set of one or more automated rules. For example, the data pre-processor can process or clean the clinical variables using expert opinion and medical knowledge in which, for example, extreme values in physiological variables can be removed. As another example, the data pre-processor can identify and remove outliers among the variables, such as replacing the top and bottom 1% of data using random uniform values generated from the neighboring data, among other options. The data pre-processor or data processing module can also comprise missing data imputation or an imputation module that performs missing data imputation. For example, according to just one embodiment, the system can replace missing nominal variables with distinct ‘missing’ category and missing continuous variables will be replaced by median values. Data imputation may be in whole or in part dependent upon the classification model utilized for the resource allocation system. The outcome of the feature processing is a set of features related to monitoring liability for each of the plurality of previously monitored patients, which thus comprises a training data set that can be utilized to train the classifier.

At 240, the training data set is utilized to train the classifier to classify a patient in one of a plurality of monitoring liability classes ranging from low monitoring liability to high monitoring liability, based at least in part on a predicted monitoring liability for the patient. The classifier can be any machine learning classifier sufficient to utilize the type of input data provided. Thus, at 250, the system comprises a trained monitoring liability classifier configured to classify a patient into one of a plurality of monitoring liability classes.

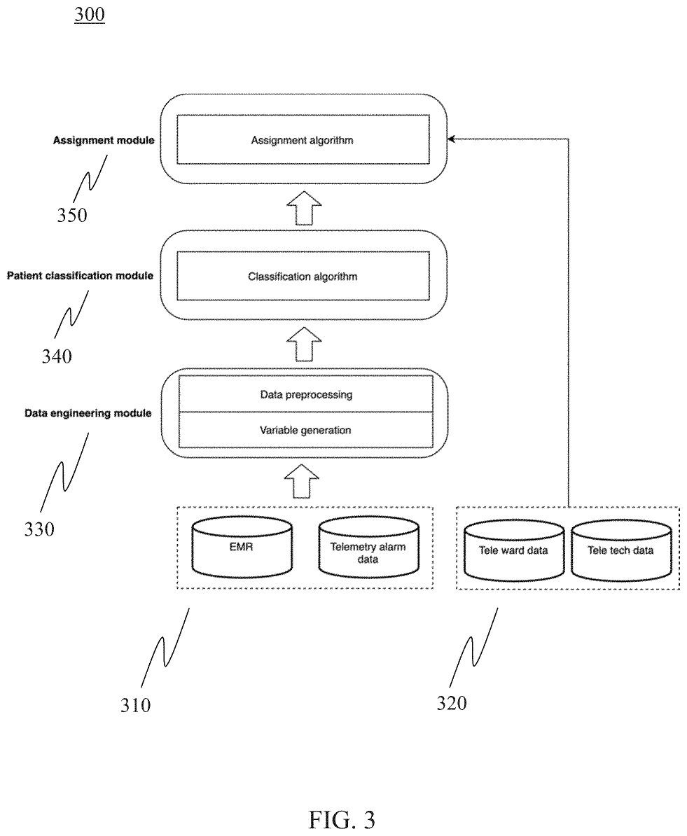

Referring to FIG. 3, in one embodiment, is a schematic representation 300 of a method for optimizing the distribution of patients among monitoring healthcare professionals. At 310, the resource allocation system receives information about a plurality of patients being monitored by a plurality of healthcare professionals. The information may comprise any information about the plurality of patients, including but not limited to medical information which will be informative for monitoring liability. The database may be an electronic medical record database, a telemetry database, and/or any other database comprising the necessary information.

At 320, the system receives information about a monitoring load for each of the plurality of healthcare professionals at the monitoring center. The information may comprise any information about the monitoring healthcare professionals, including but not limited to information about the number of patients a professional is currently monitoring, the professional's current workload, the professional's current shift info, the professional's experience, the professional's scheduling preferences, the center's staffing targets or other information about the monitoring center, and much more.

At 330, a data engineering module of the resource allocation system processes the received information such that it can be utilized by the resource allocation system to train a classifier. For example, the system may identify, extract, and process one or more variables for each patient in a dataset. The system may also engage in other data preprocessing in order generate variables that can be utilized to train the classifier.

At 340, the system trains the classifier as described or otherwise envisioned herein. Once the classifier is trained, it can be utilized to classify one or more of the plurality of patients into a monitoring liability class. According to one embodiment, the classifier is trained to classify a patient into one of a plurality of different possible monitoring liability classes. For example, the plurality of different possible monitoring liability classes may comprise classes ranging from low monitoring liability to high monitoring liability, among other options.

At 350, an assignment module of the resource allocation system determines a distribution of the plurality of patients for monitoring among the plurality of healthcare professionals based on both the received monitoring load for each of the plurality of healthcare professionals and the monitoring liability class for each of the plurality of patients. The assignment module is configured to optimize the monitoring load for each of the plurality of healthcare professionals.

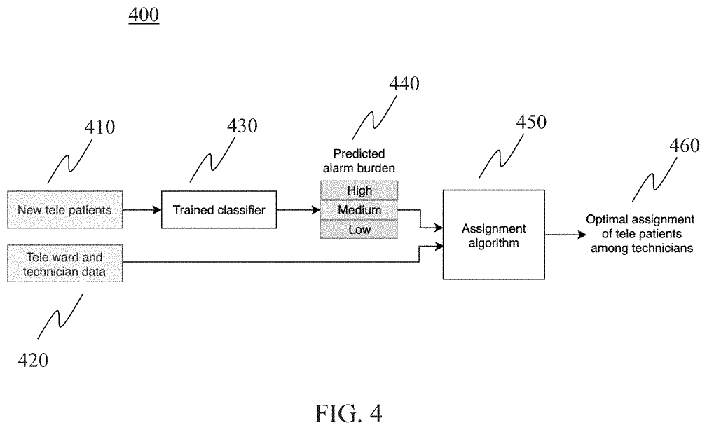

Referring to FIG. 4 is a schematic representation 400 of a method for optimizing the distribution of patients among monitoring healthcare professionals. At 410, the resource allocation system receives information about a plurality of patients being monitored by a plurality of healthcare professionals. The information may comprise any information about the plurality of patients, including but not limited to medical information which will be informative for monitoring liability. The database may be an electronic medical record database, a telemetry database, and/or any other database comprising the necessary information. At 420, the system receives information about a monitoring load for each of the plurality of healthcare professionals at the monitoring center. The information may comprise any information about the monitoring healthcare professionals, including but not limited to information about the number of patients a professional is currently monitoring, the professional's current workload, the professional's current shift info, the professional's experience, the professional's scheduling preferences, the center's staffing targets or other information about the monitoring center, and much more.

At 430, the trained classifier utilizes data from the received information about the monitored or to-be-monitored patients to assign each patient to a monitoring liability class. For example, according to the embodiment in FIG. 4, the monitoring liability classes 440 comprise “Low Predicted Alarm Burden,” “Medium Predicted Alarm Burden,” and “High Predicted Alarm Burden.”

At 450, an assignment module of the resource allocation system determines a distribution of the plurality of patients for monitoring among the plurality of healthcare professionals based on both the received monitoring load for each of the plurality of healthcare professionals and the monitoring liability class for each of the plurality of patients. The assignment module is configured to optimize the monitoring load for each of the plurality of healthcare professionals.

At 460, the assignment module of the resource allocation system re-distributes the plurality of patients for monitoring among the plurality of healthcare professionals according to the determined distribution. For example, the assignment module may comprise control of patient assignments, or may send a determined distribution to a control module for making changes to a current assignment list.

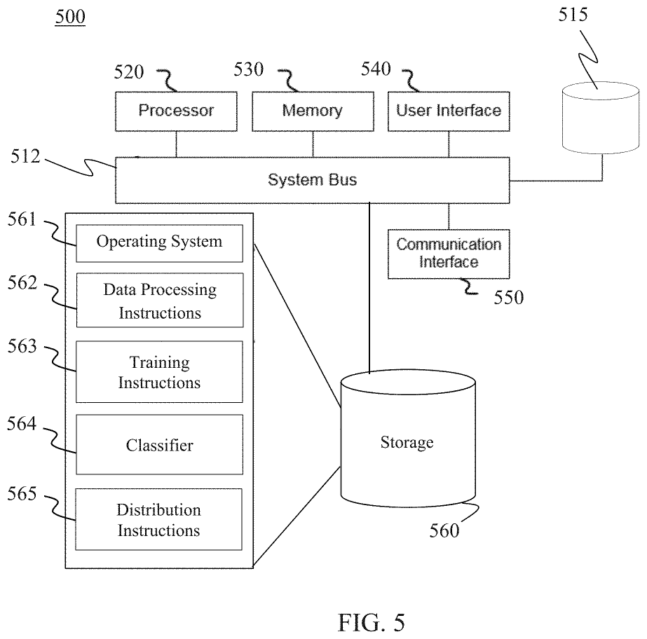

Referring to FIG. 5, in one embodiment, is a schematic representation of a system 500 for optimizing the distribution of patients among monitoring healthcare professionals. System 500 may be any of the systems described or otherwise envisioned herein, and may comprise any of the components described or otherwise envisioned herein.

According to an embodiment, system 500 comprises one or more of a processor 520, memory 530, user interface 540, communications interface 550, and storage 560, interconnected via one or more system buses 512. It will be understood that FIG. 5 constitutes, in some respects, an abstraction and that the actual organization of the components of the system 500 may be different and more complex than illustrated.

According to an embodiment, system 500 comprises a processor 520 capable of executing instructions stored in memory 530 or storage 560 or otherwise processing data to, for example, perform one or more steps of the method. Processor 520 may be formed of one or multiple modules. Processor 520 may take any suitable form, including but not limited to a microprocessor, microcontroller, multiple microcontrollers, circuitry, field programmable gate array (FPGA), application-specific integrated circuit (ASIC), a single processor, or plural processors.

Memory 530 can take any suitable form, including a non-volatile memory and/or RAM. The memory 530 may include various memories such as, for example L1, L2, or L3 cache or system memory. As such, the memory 530 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 500. It will be apparent that, in embodiments where the processor implements one or more of the functions described herein in hardware, the software described as corresponding to such functionality in other embodiments may be omitted.

User interface 540 may include one or more devices for enabling communication with a user. The user interface can be any device or system that allows information to be conveyed and/or received, and may include a display, a mouse, and/or a keyboard for receiving user commands. In some embodiments, user interface 540 may include a command line interface or graphical user interface that may be presented to a remote terminal via communication interface 550. The user interface may be located with one or more other components of the system, or may located remote from the system and in communication via a wired and/or wireless communications network.

Communication interface 550 may include one or more devices for enabling communication with other hardware devices. For example, communication interface 550 may include a network interface card (NIC) configured to communicate according to the Ethernet protocol. Additionally, communication interface 550 may implement a TCP/IP stack for communication according to the TCP/IP protocols. Various alternative or additional hardware or configurations for communication interface 550 will be apparent.

Storage 560 may include one or more machine-readable storage media such as read-only memory (ROM), random-access memory (RAM), magnetic disk storage media, optical storage media, flash-memory devices, or similar storage media. In various embodiments, storage 560 may store instructions for execution by processor 520 or data upon which processor 520 may operate. For example, storage 560 may store an operating system 561 for controlling various operations of system 500.

It will be apparent that various information described as stored in storage 560 may be additionally or alternatively stored in memory 530. In this respect, memory 530 may also be considered to constitute a storage device and storage 560 may be considered a memory. Various other arrangements will be apparent. Further, memory 530 and storage 560 may both be considered to be non-transitory machine-readable media. As used herein, the term non-transitory will be understood to exclude transitory signals but to include all forms of storage, including both volatile and non-volatile memories.

While system 500 is shown as including one of each described component, the various components may be duplicated in various embodiments. For example, processor 520 may include multiple microprocessors that are configured to independently execute the methods described herein or are configured to perform steps or subroutines of the methods described herein such that the multiple processors cooperate to achieve the functionality described herein. Further, where one or more components of system 500 is implemented in a cloud computing system, the various hardware components may belong to separate physical systems. For example, processor 520 may include a first processor in a first server and a second processor in a second server. Many other variations and configurations are possible.

According to an embodiment, system 500 may comprise or be in remote or local communication with a database or data source 515. Database 515 may be a single database or data source or multiple. Database 515 may comprise input data utilized to train the monitoring liability classifier. The input data can comprise a wide variety of input types in addition to the monitoring liability of a patient. As an example, the input data can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or telemetry alarm related data such as arrhythmia-related alarms (ventricular tachycardia, ventricular bradycardia, atrial fibrillation, asystole, and more), physiologic alarms such as heart rate, respiratory rate, apnea, SpO₂, invasive arterial pressure, noninvasive blood pressure, and more, and/or technical alarms such as artifact, ECG leads fail, respiratory leads fail, blood pressure sensor fail, and more. Many other types of input data are possible.

According to an embodiment, database 515 may comprise information about a plurality of patients being monitored or to-be-monitored. The information may comprise any information about the plurality of patients, including but not limited to medical information which will be informative for monitoring liability. Preferably, the received information comprises input data related to the training data for the trained classifier utilized by the system, to maximize the accuracy of the classifier.

According to an embodiment, database 515 may comprise information about a monitoring load for a plurality of healthcare professionals. The information may comprise any information about the monitoring healthcare professionals, including but not limited to information about the number of patients a professional is currently monitoring, the professional's current workload, the professional's current shift info, the professional's experience, the professional's scheduling preferences, the center's staffing targets or other information about the monitoring center, and much more.

According to an embodiment, storage 560 of system 500 may store one or more algorithms and/or instructions to carry out one or more functions or steps of the methods described or otherwise envisioned herein. For example, processor 520 may comprise one or more of data processing instructions 562, training instructions 563, classifier 564, and/or distribution instructions 565.

According to an embodiment, data processing instructions 562 direct the system to retrieve and process input data used by training instructions 563 to generate a trained classifier 564. The data processing instructions 562 direct the system to receive or retrieve input data comprising medical information about a plurality of monitored patients. The medical information includes at least the monitoring liability for each of the monitored patients. As described above, the input data can comprise a wide variety of input types in addition to the monitoring liability of a patient.

The data processing instructions 562 also direct the system to process the input data to generate a plurality of features related to monitoring liability for each of the plurality of previously monitored patients. This can be accomplished by a variety of embodiments for feature identification, extraction, and/or processing. For example, a data transformer or transformer module can read input data and generate predictor and outcome variables. As an example, alarm rates at the patient level can be used as the outcome variable for model training. A data pre-processor or data processing module can process the predictor and outcome variables using a set of one or more automated rules. The outcome of the feature processing is a set of features related to monitoring liability for each of the plurality of previously monitored patients, which thus comprises a training data set that can be utilized to train the classifier.

According to an embodiment, training instructions 563 direct the system to utilize the processed data to train the classifier to classify a patient in one of a plurality of monitoring liability classes ranging from low monitoring liability to high monitoring liability, based at least in part on a predicted monitoring liability for the patient. The classifier can be any machine learning classifier sufficient to utilize the type of input data provided. Thus, the system comprises a trained monitoring liability classifier 564 configured to classify a patient into one of a plurality of monitoring liability classes.

According to an embodiment, distribution instructions 565 direct the system to determine a distribution of the plurality of patients for monitoring among the plurality of healthcare professionals based on both the received monitoring load for each of the plurality of healthcare professionals and the monitoring liability class for each of the plurality of patients. The distribution instructions are configured to optimize the monitoring load for each of the plurality of healthcare professionals. According to just one embodiment, the distribution instructions use predicted alarm burden classes generated by the patient classification module and optimize the distribution of patients among telemetry technicians. The data about tele-ward and technicians—such as number of patients currently monitoring, current workload, current shift info, experience, and others—can also be used as inputs to optimize scheduling.

The distribution instructions 565 also direct the system to re-distribute the plurality of patients for monitoring among the plurality of healthcare professionals according to the determined distribution. For example, distribution instructions may comprise control of patient assignments, or may send a determined distribution to a control module for making changes to a current assignment list.

According to an embodiment, the resource allocation system is configured to process many thousands or millions of datapoints in the input data, the received to-be-monitored patient data, and/or the received healthcare professional monitoring burden data. For example, generating a functional and skilled trained classifier using an automated process such as feature identification and extraction and subsequent training requires processing of millions of datapoints from input data and the generated features. This can require millions or billions of calculations to generate a novel trained classifier from those millions of datapoints and millions or billions of calculations. As a result, each trained classifier is novel and distinct based on the input data and parameters of the machine learning algorithm. Thus, generating a functional and skilled trained classifier comprises a process with a volume of calculation and analysis that a human brain cannot accomplish in a lifetime, or multiple lifetimes.

Similarly, the resource allocation system can be configured to continually receive data, classify patients, and redistribution the patients to optimize healthcare professional workload. This requires the analysis of thousands or millions of datapoints on a continual basis to optimize distribution, requiring a volume of calculation and analysis that a human brain cannot accomplish in a lifetime.

By optimize healthcare professional workload, this novel resource allocation system has an enormous positive impact on healthcare compared to prior art systems. As just one example, by optimizing healthcare professional workload, the system reduces alarm fatigue in healthcare professionals. This alarm fatigue directly impacts technicians, leading to annoyance, anxiety, low job satisfaction, and burn out. Alarm fatigue can lead technicians to ignore alarms, disable alarms inappropriately, or delay a response. All of these actions could pose a significant threat to patient safety, including prolonging hospital stays and healthcare costs, and in the most serious cases leading to adverse clinical outcomes or even patient death. By reducing alarm fatigue, healthcare professionals are able to provide improved care, leading to significantly improved patient outcomes.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for allocating resources using a resource allocation system, the method comprising:
receiving, by the resource allocation system, information about a plurality of patients being monitored by a plurality of healthcare professionals;
receiving, by the resource allocation system, information about a monitoring load for each of the plurality of healthcare professionals;
classifying, by a trained monitoring liability classifier of the resource allocation system, each of the plurality of patients into one of a plurality of monitoring liability classes;
determining, by an assignment module of the resource allocation system, a distribution of the plurality of patients for monitoring among the plurality of healthcare professionals based on both the received monitoring load for each of the plurality of healthcare professionals and the monitoring liability class for each of the plurality of patients, wherein the distribution optimizes the monitoring load for each of the plurality of healthcare professionals; and
redistributing, by the assignment module of the resource allocation system, the plurality of patients for monitoring among the plurality of healthcare professionals according to the determined distribution.

2. The method of claim 1, further comprising the steps of:
receiving new information about the plurality of patients and/or the monitoring load for each of the plurality of healthcare professionals;
determining an updated distribution of the plurality of patients for monitoring among the plurality of healthcare professionals based on the received new information; and
redistributing the plurality of patients based on the determined updated distribution.

3. The method of claim 1, further comprising the steps of:
receiving, by the resource allocation system, input data comprising medical information about a plurality of monitored patients, the information comprising a monitoring liability for each of the plurality of monitored patients;
generating, by a feature processing module of the resource allocation system, a plurality of features related to monitoring liability for each of the plurality of monitored patients; and
training, using the plurality of features, a classifier to classify a patient in one of a plurality of monitoring liability classes, based at least in part on a predicted monitoring liability for the patient.

4. The method of claim 1, wherein the information about a plurality of patients being monitored by a plurality of healthcare professionals comprises one or more of demographic information, health history, treatment data, telemetry data, and/or diagnosis data for one or more of the plurality of patients.

5. The method of claim 1, wherein the information about a monitoring load for each of the plurality of healthcare professionals comprises one or more of a number of patients being monitored by the healthcare professional, and/or an experience level of the healthcare professional for one or more of the healthcare professionals.

6. The method of claim 1, wherein monitoring liability comprises an expected or predicted number of alarms for a patient during a time period.

7. The method of claim 1, wherein monitoring liability comprises a severity of one or more of an expected or predicted number of alarms for a patient during a time period.

8. The method of claim 1, wherein classifying comprises a prediction of a monitoring liability for at least one of the plurality of patients.

9. The method of claim 1, wherein the plurality of monitoring liability classes comprises at least a low monitoring liability class and a high monitoring liability class.

10. A resource allocation system configured to allocate resources, comprising:
data comprising information about a plurality of patients being monitored by a plurality of healthcare professionals;
data comprising information about a monitoring load for each of the plurality of healthcare professionals;
a monitoring liability classifier trained to classify each of the plurality of patients into one of a plurality of monitoring liability classes; and
a processor configured to: (i) direct the classifier to classify each of the plurality of patients into one of the plurality of monitoring liability classes; (ii) determine a distribution of the plurality of patients for monitoring among the plurality of healthcare professionals based on both the received monitoring load for each of the plurality of healthcare professionals and the monitoring liability class for each of the plurality of patients, wherein the distribution optimizes the monitoring load for each of the plurality of healthcare professionals; and (iii) redistribute the plurality of patients for monitoring among the plurality of healthcare professionals according to the determined distribution.

11. The system of claim 10, wherein the processor is further configured to: receive new information about the plurality of patients and/or the monitoring load for each of the plurality of healthcare professionals; determine an updated distribution of the plurality of patients for monitoring among the plurality of healthcare professionals based on the received new information; and redistribute the plurality of patients based on the determined updated distribution.

12. The system of claim 10, wherein the processor is further configured to: receiving input data comprising medical information about a plurality of monitored patients, the information comprising a monitoring liability for each of the plurality of monitored patients; generate a plurality of features related to monitoring liability for each of the plurality of monitored patients; and train the classifier to classify a patient in one of a plurality of monitoring liability classes, based at least in part on a predicted monitoring liability for the patient.

13. The system of claim 10, wherein the information about a plurality of patients being monitored by a plurality of healthcare professionals comprises one or more of demographic information, health history, treatment data, telemetry data, and/or diagnosis data for one or more of the plurality of patients.

14. The system of claim 10, wherein the information about a monitoring load for each of the plurality of healthcare professionals comprises one or more of a number of patients being monitored by the healthcare professional, and/or an experience level of the healthcare professional for one or more of the healthcare professionals.

15. The system of claim 10, wherein monitoring liability comprises an expected or predicted number of alarms for a patient during a time period, and/or a severity of one or more of an expected or predicted number of alarms for a patient during a time period.
